(19) United States
(12) Patent Application Publication
(10) Pub. No.: US 2003/0088307 A1
(43) Pub. Date: May 8, 2003

(54) Potent coatings for stents

(76) Inventors: John E. Shulze, Rancho Santa Margarita, CA (US); Ronald E. Betts, La Jolla, CA (US); Douglas R. Savage, Del Mar, CA (US)

Correspondence Address: J.C. Patents, Suite 250, 4 Venture, Irvine, CA, 92618, US

(21) Appl. No.: 10/150,909

(22) Filed: May 16, 2002

Related U.S. Application Data
(60) Provisional application No. 60/337,970, filed on Nov. 5, 2001.

(51) Int. Cl. A61F 2/06

(52) U.S. Cl. 623/1.15; 623/1.42; 427/2.25

(57) ABSTRACT

A stent having an expandable stent body with a generally tubular shape comprises a series of support surfaces upon which a polymer stent coating has been applied. One or more bioactive agents are disposed within the coating. The coating is applied by evaporating solvent from a solution which has been applied to the stent surfaces from a pressurized reservoir or positive displacement pumping means attached to a delivery tube. The delivery tube's longitudinal or X-Y-Z position along the body of the stent, the rotation of the stent along its longitudinal axis, and the delivery rate are coordinated by a programmable controller to deposit precise and repeatable amounts of polymer and agent on the stent surfaces. Preferably, an anti-restenosis agent consisting of a potent analogue or derivative of tranilast are disposed in a bioerodable stent coating, comprising poly(lactic acid), or, alternatively, in a biodurable stent coating comprising EVA.

This application claims the benefit under 35 U.S.C. 119(e) of the filing date of Provisional Application No. 60/337,970, filed on Nov. 5, 2001, and expressly incorporated herein by reference.

### BACKGROUND OF THE INVENTION

The present application is generally related to medical devices. More specifically, the present invention relates to stent coatings capable of releasing bioactive agents over time into tissue, usually a blood vessel wall or another vascular conduit wall that is being supported by the stent structure. The coating preferably includes one or more bioactive agents found to be useful for the control of restenosis or to reduce late thrombus formation.

A stent is a type of endovascular implant, usually generally tubular in shape, and usually with a cylindrical outer diameter which is expandable to be permanently inserted into a blood vessel to provide mechanical support to the vessel and to maintain or re-establish a flow channel during or following angioplasty. The support structure of the stent is designed to prevent early collapse of a vessel which has been weakened and damaged by angioplasty, thereby minimizing negative remodeling and spasm of the vessel while the healing of the damaged vessel wall proceeds over a period of weeks or months. During the healing process, inflammation caused by the angioplasty and implant injury may cause smooth muscle cell proliferation and regrowth inside the stent, thus partially closing the flow channel, and reducing or eliminating the beneficial effect of the angioplasty/stenting procedure. This process is called restenosis. Blood clots may also form inside the newly implanted stent due to the thrombotic nature of the stent surfaces, even when so-called biocompatible materials are used to form the stent. While large blood clots may not form during the angioplasty procedure itself or immediately post-procedure due to the current practice of peri-procedural injection of powerful anti-platelet drugs into the bloodstream, some thrombosis is always present, at least on a microscopic level on stent surfaces, and it is thought to play a significant role in the early stages of restenosis by establishing a biocompatible matrix on the surfaces of the stent wherein smooth muscle cells may subsequently attach and multiply.

Stent coatings are known which contain bioactive agents (for example, drugs) which are claimed to act to reduce or eliminate thrombosis or restenosis. Such bioactive agents may be dispersed or dissolved in either a bio-durable or bio-erodable polymer matrix which is attached to the surface of the stent prior to implant. Methods have been described for preparing solutions of polymers in solvents, with the bioactive agent dissolved or dispersed in the solution. After spraying, casting, or dipping the stent into the polymer solution, the solvent is allowed to evaporate, leaving a layer of polymer on the surface of the stent, referred to as a stent coating (U.S. Pat. Nos. 6,153,252 and 5,980,972 disclose examples of such approaches). U.S. Pat. No. 5,578,075 describes in one alternative method the pouring of a polymer/solvent/drug mixture over a stent while the stent is being rotated. In another approach, described in European Patent Specification No. 0627226 B1, a method has been described for “dropping by means of a pipette” the polymer solution, while the stent is rotated at a constant rpm about its longitudinal axis using a gearmotor until complete coating of the stent occurs. Methods have been described for casting the polymer solution in sheet form onto a surface, whereupon the solvent is allowed to evaporate, leaving a layer of the polymer coating laminated to the sheet surface. The resulting laminated sheet is subsequently cut and formed into the tubular shape of a stent. Such a procedure is described, for example, in U.S. Pat. No. 5,649,977. Stent coatings are also known where two precursors are polymerized directly on the stent surface in the presence of the bioactive agent, or where the polymer/drug melt solution is extruded over the surfaces of the stent. Of course, it is also known to place a separate, flexible and expandable polymer sleeve over the stent structure to act as a drug delivery reservoir, and further to create cavities or porosity in such polymer sleeves for storage and subsequent elution of agent. It is additionally known to make a coating on such a stent by vapor or plasma deposition of a polymer which, in a second step, is capable of absorbing and storing an agent when placed in a solvent solution of the agent. Finally, it is known to make a stent completely from a bioerodable polymer, with a bioactive agent dissolved into the polymer during a casting or dipping process, as described in U.S. Pat. Nos. 5,443,458 and 5,935,506, as well as in European Patent Application No. 281482, and Japanese Patent Application No. 11137694A2.

Regardless of which process is used to form or attach the polymer and bioactive agent to the stent structure, after implant, the bioactive agent is expected to diffuse out of the polymer matrix and preferably into the surrounding tissue over a period of weeks or months, ideally matching the time course of the process of restenosis. If the polymer is bioerodable, in addition to the process of diffusion, the bioactive agent may also be released as the polymer degrades or dissolves, making the agent more readily available to the surrounding tissue environment. Bioerodable stents and biodurable stents are known where the outer surfaces or even the entire bulk of polymer material is porous. For example, PCT Publication No. WO 99/07308, which is commonly owned with the present application, discloses such stents, and is expressly incorporated by reference herein. When bioerodable polymers are used as drug delivery coatings, porosity is variously claimed to aid tissue ingrowth, make the erosion of the polymer more predictable, or to regulate or enhance the rate of drug release, as, for example, disclosed in U.S. Pat. Nos. 6,099,562, 5,873,904, 5,342,348, 5,873,904, 5,707,385, 5,824,048, 5,527,337, 5,306,286, and 6,013,853.

In addition to stent coatings containing bioactive agents in solution or in a dispersion within the polymer, stent coatings are known which contain bioactive agents in particle form, as disclosed in, for example, U.S. Pat. No. 5,605,696. U.S. Pat. No. 5,447,724 describes a multilayer drug delivery coating wherein the outer layer is a drug diffusion controlling layer, and in one embodiment the diffusion controlling layer contains particles which may be a water soluble antiplatelet agent (i.e heparin or aspirin), which dissolves readily when the device is placed in vivo to create pores in the diffusion controlling outer layer of the drug delivery coating. After the particle layer in the diffusion controlling outer layer dissolves, the device begins to deliver over an extended period an agent stored in a polymer reservoir layer located underneath the diffusion controlling layer.

Metallic or non-polymer coated stents are known which contain grooves, recesses, or pores where drugs can be stored, either mixed with polymer, or alone. For example, such stents are disclosed in U.S. Pat. Nos. 5,843,172, 5,972,027, 5,902,266, 5,891,108, 5,972,027, 5,163,958, and 6,273,908.

Again with respect to polymer coating of stents, it is known to attach a primer coat of pure polymer without agent to the stent surface, to increase adhesion of a subsequent polymer layer containing an agent, as disclosed in U.S. Pat. No. 5,837,313, or to make coatings of multiple layers with different agents in each layer, or different concentrations of the same agent in different layers to tailor the drug elution characteristics of the device to any perceived need, as disclosed in U.S. Pat. Nos. 5,837,313, 6,258,121, and 5,824,048.

Although many stents containing drug delivery apparatus have thus been devised, to make these devices effective, there remains a great need to identify drug agents which can control the unfavorable processes of thrombosis and restenosis, and thus prevent reduction in the therapeutic benefit of the devices. Not all of the described devices are compatible with all of the agents that are theoretically useful, either because the agent may not be soluble in a common solvent or because the drug has other physical attributes (for example, it breaks down at polymer processing temperatures) which prevents it from being incorporated into the selected polymer forming or coating process, or, alternatively because the agent may not be sufficiently potent in the amounts that can be contained in the agent storage reservoir of the stent, however designed and specified, to achieve a therapeutic effect in vivo. A further consideration is the diffusion rate of the drug through the chosen polymer. If the drug is a large protein, it will have to be incorporated into a polymer matrix which allows the diffusion of such large molecules to achieve a suitable diffusion rate. Thus some drugs are not suitable for effective diffusion out of polymers with small openings within the matrices. Furthermore, the mechanisms by which many of the agents interact with the cell reproductive machinery or blood clotting cascade in vivo is often poorly understood, so it is difficult to predict strictly on the basis of cell culture results, small animal experiments, or chemical makeup as to which agents (drugs) will be effective on drug delivery stents designed to be placed in humans or other large animals. It is important to note that a number of the bioactive agents that have been shown effective to limit restenosis in small animal models such as the rat have been later to been shown to be ineffective in humans or large animals. Thus the selection of a suitable drug agent is largely, at the current state of the art, a laborious process of experimentation with rare success.

Heparin, as well as other antiplatelet or antithrombolytic surface coatings, are known which are chemically bound to the surface of the stent to reduce thrombosis. A heparinized surface is known to interfere with the blood clotting cascade in humans, preventing attachment of platelets (a precursor to thrombin) on the stent surface. Stents have been described which include both a heparin surface and an active agent stored inside of a coating (see U.S. Pat. Nos. 6,231,600 and 5,288,711, for example).

Agents for use in a drug delivery stent have been described which are believed to be effective against restenosis. U.S. Pat. No. 6,159,488 describes the use of a Quinazolinone derivative. U.S. Pat. No. 6,171,609 describes the use of a cytoskeltal inhibitor-Taxol. U.S. Pat. No. 5,176,981 cites the use of Paclitaxel, a cytotoxic agent thought to be the active ingredient in the agent Taxol. The metal silver is cited in U.S. Pat. No. 5,873,904. Tranilast, a membrane stabilizing agent thought to have anti-inflammatory properties is used in U.S. Pat. No. 5,733,327. Usage of Rapamycin, an immunosuppressant reported to suppress both smooth muscle cell and endothelial cell growth is described in U.S. Pat. Nos. 5,288,711 and 6,153,252. In addition, many other potentially useful drugs, of various chemical makeups, are mentioned in the other referenced patents. PCT Publication No. W/O 97/35575 describes the administration of rapamycin derivatives to treat restenosis induced by angioplasty, but not the administration of these compounds from an implantable device such as a stent.

In FIG. 1 is shown a state of the art metallic, corrugated ring stent 10 for use in the coronary arteries of a human. In its contracted state, prior to deployment in the artery, it has a tubular diameter of approximately 0.75 mm. In FIG. 2 is shown the same stent, which has been cut longitudinally and unrolled onto a flat surface so that the outer surface pattern of the stent can be more clearly seen. The stent is made up of a series of expandable bands (rings) arrayed in series along a longitudinal axis to form a tubular stent structure. The individual stent rings 12 are made up of alternating bendable elements (bend joints 14) and non-bendable straight elements (struts 16), which are connected together in an undulating, or “corrugated” manner. For coronary artery applications, the struts are approximately 1 mm in length, and viewed in FIG. 2A, at cross-section A-A′ of FIG. 2, the stent struts are typically rectangular in shape, with approximate dimensions of 0.001-0.007″ thick, and 0.003-0.007″ wide. These approximate dimensions might vary for stents used in other areas of the body, generally in proportion to the size of the vascular conduit being treated. The individual rings are flexibly attached to each other through a series of bendable links 18. This type of stent structure has been shown to have high flexibility for catheter insertion and deployment in small, tortuous vessel anatomies, while maintaining good vessel support characteristics and a moderate restenosis rate. As a result, variants of this design are in wide use.

However, when it is decided to apply a coating to this type of stent, there is a problem with trying to use a spraying or dipping process. The action of dipping or spraying the stent suggests that fluid will contact the top and side surfaces in a non selective manner. Specifically, as the spray or dipping is applied, the polymer solution will tend to be deposited unevenly, with droplets tending to form where the stent elements are closer together. As the solvent evaporates, more polymer and agent will be deposited near bend joints, with relatively less in the straight sections (struts). This is exactly opposite of what is desired, as the bend joints undergo large deformations once the stent is expanded. With thick layers of polymer being deposited in the corners and bend joints of the stent, the possibility of surface cracking or flaking of the polymer coating is increased. The pooling of the polymer in certain areas of the stent topography during spraying or dipping also causes uneven surface distribution of agent, so not all of the areas of the vessel wall may be equally treated with agent. This is a serious problem if the agent has a narrow therapeutic window between a delivered tissue concentration which achieves therapeutic effect and a tissue concentration which is toxic. U.S. Pat. No. 6,156,373 goes into considerable detail about the process problems associated with achieving an even distribution of polymer using brushing, rolling, dipping and spraying. The uneven distribution of polymer and agent caused by these processes may be reduced, but not eliminated, by applying multiple thin coats, with careful solvent evaporation between coats. But, such an approach increases the cost of the coating process. Thus, it would be highly desirable to develop a coating process where the amount of polymer/agent solution deposited in each area of the stent structure could be programmed and precisely controlled. Precise agent deposition control would be of even further benefit in applications where it may be desirable to apply more medication to certain regions of the stent. One example would be the treatment of a vessel which has undergone much greater injury at one area, perhaps an end of the stent that has a dissection, or in an area of severe tearing of the vessel wall. Such severe injuries have been shown to lead to greater restenosis in the vicinity of the injury. It would be logical to deploy more medication, or perhaps multiple medications in the region of the severe injury. Another example is an area of the vessel wall which contains a calcium or soft plaque deposit. Depending on the mechanism of action of the agent, it may be desirable to deliver a greater or lesser degree of agent to the area of the plaque deposit.

The prior art processes of casting, extrusion, or placing of an expandable sleeve over the stent are suitable if it is desired to create a solid polymer barrier between the stent and the tissue wall. However, in our experiments in pigs, placement of a sealing membrane between the internal lumen of the stent and the vessel wall may lead to a foreign body reaction, even when using highly biocompatible surfaces to form the sleeve. The process by which this foreign body reaction is triggered is not understood, but it is theorized that the blood flowing through the interior of the stent lumen requires one or more biochemical signals expressed by the tissue wall to prevent triggering of such a reaction.

In known stents having surface coatings of heparin, there is also a significant problem of fouling of the heparin surface over time. Although chemically bonded coatings of heparin have high activity for the prevention of thrombus during the initial days after implantation, it has been found that the effect is not durable. As time passes, the heparin surface becomes compromised, the heparin compound loses activity due to reactions with substances within the blood, or is lost to the blood circulation, and thrombus begins to form. Possibly for this reason, heparin-coated stents alone have not been shown effective for the prevention of restenosis.

While some of the known bioactive agents for use on stents may strikingly reduce restenosis, they may, at the same time, cause delayed healing of the vessel intima. So long as the vessel intima is not fully healed, the stent surfaces have not been fully covered with viable living endothelial tissue, and they remain thrombogenic to some degree. This sets the stage for “late” thrombosis, i.e. thrombosis occurring months or years after implantation of the stent. This is a dangerous and potentially life threatening situation, should a thrombosis form and be severe enough to lead to vessel blockage and heart attack when the patient has long since left the hospital.

Accordingly, there remains a need for an improved stent coating which is fast and inexpensive to apply, provides an optimum, programmable distribution of polymer and agent on the support structure of the stent, and which acts to control restenosis caused by angioplasty and stent implantation injury. There is also a need for an improved stent coating which can reduce in-stent thrombosis not just immediately following the implant procedure, but preferably continues to exhibit at least some antithrombotic effect in the weeks and months after stent implant. There is further a need to discover new agents which are potent enough to be effective when applied in very thin coatings. Such thin coatings would be insertable into small tortuous vessels, and would work effectively with porous and non-porous bio-erodable polymer coatings of the current stent art such as poly(L-lactic acid), poly(D,L-lactic acid), e-caprolactone, polyglycolic acid, co-polymers thereof, and the like. Bioerodable polymers are desirable because they do not remain in the tissue after the drug has been delivered. However, because they are able to dissolve, they are at least to some extent, hydrophilic, yet two of the agents known to have high anti-proliferative properties in humans and frequently cited for use in reduction of restenosis, specifically paclitaxel and rapamycin, are highly hydrophobic, and thus a poor match for a bio-erodable stent coating. In the article entitled “Physiological Transport Forces Govern Drug Distribution for Stent-Based Delivery”, Hwang, et al, Circulation, July 2001, the advantages and disadvantages of more hydrophilic drugs are discussed for use on a drug eluting stent. As described by Hwang in the above-referenced article, drugs with greater solubility (more hydrophilic drugs) will partition more evenly in tissue space and thus may provide a more uniform therapeutic effect, however, the accompanying disadvantage of more soluble drugs is that the peak concentration in tissue walls is reduced. Thus it is desirable to find a drug of high potency, so that the potential benefit of more uniform tissue treatment can be achieved in a low profile device using a thin coating.

It would thus be desirable to find agents that are more hydrophilic to more closely match the breakdown characteristics of bioerodable stents. And further, it would be highly desirable to make stent coatings that provide less tissue toxicity, and an improved healing of the vessel after treatment. Many cytoxic agents of the current art are toxic to all of the cell types acting in or on the vessel wall-not just the smooth muscle cell. Thus, it would be desirable to find agents for incorporation into drug delivery stents that are specifically adapted to control smooth muscle cell proliferation and migration, while not creating toxins which affect endothelial cell proliferation and other cellular processes which are so highly important to a rapid vessel wall healing, and which additionally return homeostasis to the vessel and render the tissue at the site of prior injury non-thrombogenic.

### SUMMARY OF THE INVENTION

The present invention includes a stent having an expandable stent body having a generally tubular shape. The stent comprises a series of support surfaces upon which a stent coating, such as a polymer coating, has been applied. The stent used in the present invention can be metal or non-metal stent and can be a bioerodeable or biodurable stent. The cross section of the stent strut may have different shapes such as square, rectangular, circle, oval, or other suitable shapes and may vary from one portion to another. The invention further includes one or more bioactive agents disposed within the coating and/or, if desirable, disposed or physically/chemically attached on the surface of the coating. The coating is applied by evaporating a solvent from a solution which has been applied to the stent surfaces from a pressurized reservoir or positive displacement pumping means attached to a delivery tube. The delivery tube's longitudinal or X-Y-Z position along the body of the stent, the rotation of the stent along its longitudinal axis, and the delivery rate are coordinated by a programmable controller to deposit precise and repeatable amounts of polymer and agent on the stent surfaces. The coating of the stent is generally accomplished in a single pass of the solvent/polymer/drug dispensing system, sequentially across all of the interconnected outside surfaces of the stent support structure. If desired, however, the polymer containing the bioactive agent(s) can be selectively applied as islands of coating on the external and/or internal surfaces in similar or varying degrees of thickness. In a first embodiment, an anti-restenosis agent consisting of a potent analogue or derivative of Tranilast is disposed in a bioerodable (biodegradable) stent coating comprising poly(D,L-lactic acid), or poly(L-lactic acid), or poly(D-lactic acid), or a mixture thereof, or, alternatively, a biodurable coating comprising ethylvinyl hydroxylated acetate (EVA) or Ethylene Vinyl Alcohol (EVOH).

In a second embodiment, heparin and an anti-restenosis agent comprising a potent analogue or derivative of Tranilast are disposed in a bioerodable stent coating comprising poly(lactic acid), such as poly(D,L-lactic acid), poly(L-lactic acid), poly(D-lactic acid), or a mixture thereof. Heparin is present in crystalline form in the coating, and as it dissolves into the blood, acts to increase the porosity of the stent surface over time. This causes the poly(lactic acid) to erode more rapidly, releasing additional amounts of heparin and the agent, causing the active agent and heparin to be available in the vessel during the entire period of vessel healing.

More particularly, in one aspect of the invention there is provided a stent comprising an expandable stent body having a generally tubular shape, and a plurality of support surfaces on the stent body upon which a polymer stent coating is applied. The coating includes one or more bioactive agents disposed therein, comprising a potent analogue or derivative of Tranilast. In some embodiments, the thickness of the coating on one (or a portion thereof) of the plurality of support surfaces is different than the thickness of the coating on another one (or a portion thereof) of the plurality of support surfaces, to thereby cause a proportionate increase or decrease in bioactive agent delivery in various regions of the stent. The analogue or derivative of Tranilast selectively controls smooth muscle cell proliferation, while not interfering to any substantial degree with proliferation of endothelial cells. The polymer can be chosen from the group including Poly(D,L-lactic acid), Poly(L-lactic acid), Poly(D-lactic acid), PVA, Poly(ethylene oxide), Poly(glycolic acid), EVA, e-Caprolactone, Ethylene Vinyl Alcohol, or copolymers thereof, or mixtures thereof.

In certain embodiments, the polymer coating acts as a primary drug storage reservoir, and an antiplatelet agent is continuously eluted from the primary drug storage reservoir of the stent, which drug storage reservoir is typically a bioerodable coating.

A typical strut thickness of a low profile stent as used in the present invention is in the range of 75-125 microns and the thickness of the polymer coating surrounding the low profile strut in the present invention is usually in the range of about 3-25 μm, typically 6-10 μm. Further, the amount of polymer required to contain sufficient amounts of a compound having a 2 or 3 times lower potency on the stent would be proportionately increased. If the coating thickness were increased to, say, 30 microns per side, then in addition to adding up to 60 microns of added thickness (a 50% increase) to the strut thickness, the added bulk of coating, according to our experiments in swine, would cause inflammation of the vessel wall, partially or totally negating any therapeutic benefits of the drug.

Further, it has been discovered that only a certain amount of drug can be loaded into the polymer without compromising the structural integrity of the polymer, and in the above embodiments of the present invention this is about 50% by weight of drug (i.e. a drug/polymer weight ratio of 1 to 1). This value may vary with different polymers and bioactive agents. Thus, a further benefit of these potent derivatives is that less polymer coating is actually needed to physically contain the drug on the stent. Thus the benefits of a potent compound of the present invention are multiplied through the use of less polymer, which further reduces restenosis and stent profile, and by the ability of these potent, yet more soluble compounds to distribute more evenly within the vessel wall tissues, effecting more uniform control of restenosis.

In yet another aspect of the invention, there is provided a vascular stent containing a bioactive agent effective for the reduction of restenosis where the bioactive agent is a potent analogue or derivative of tranilast. More preferably, the bioactive agent is a diarylamide derivative of tranilast.

In another aspect of the invention, there is provided a vascular stent having a stent support structure, and containing two biologically active agents in a coating on a surface of the stent support structure, wherein one of the two agents is present in an effective quantity for control of restenosis, and the other of the two agents comprises an antiplatelet, fibrinolytic, or thrombolytic agent in soluble crystalline form, wherein the antiplatelet, fibrinolytic, or thrombolytic agent can be released for the same duration as the anti-restenosis agent. The second agent can be heparin, aspirin, hirudin, ticlopadine, urokinase, streptokinase, tissue plasminogen activator (TPA), eptifibatide, or abciximab, or a combination thereof.

In still another aspect of the invention, there is provided a fully bioerodable stent made by a process of applying to a preform a coating from a pressurized reservoir attached to a delivery tube. The tube's position along a longitudinal axis of the stent, rotation of the preform about its longitudinal axis, and pressure levels in the reservoir are coordinated by a programmable controller to uniformly or, if desirable, selectively apply the polymer which forms the body of the bioerodable stent.

In another aspect of the invention, there is disclosed a method of coating a stent comprising an expandable stent body having a generally tubular shape. The stent body comprises a plurality of support surfaces. The method comprises applying a polymer coating to the support surfaces, which coating includes one or more bioactive agents disposed within the coating and being applied from a pressurized source attached to a delivery tube. A further inventive step comprises coordinating the tube's position along a longitudinal axis of the stent, and rotation of the stent about the longitudinal axis, using a programmable controller. A vertical height of a distal end of the tube is also coordinated by the controller.

In a typical approach, the coating is a bioerodeable polymer in a solvent solution, and the solvent is permitted to evaporate after exiting the delivery tube to form the coating. Additionally, the coating step is accomplished in a single pass of a coating dispensing system, and the position of the tube is moved or stepped in small increments to sequentially apply the polymer solution across all of the interconnected outside surfaces of the stent support structure. However, it is also within the scope of the invention to apply a bioactive agent from the delivery tube which does not employ a polymer as the agent binding means of the coating. As an example, a solvent/agent mixture could be applied to the stent support structure using the delivery tube and programmable controller of the present invention, wherein the agent or agent(s) are directly chemically bound to the bare metal surface. This may be achieved by chemically activating the metal surface through application of an appropriate primer containing or a chemically reactive group, or by plasma etching the metal surface. As disclosed herein, biodurable polymers may also be used instead of bioerodeable polymers to contain the drugs and bind them to the metal stent support structure. And, finally, although the invention have been described as bioactive coatings which are applied to expandable steel stent structures of the current stent art, or as forming a part of a fully bioerodable stent structure, it should be appreciated that the coatings could also be applied to other types of stent structures, for example those of self-expanding stents, for example the nitinol types which currently find utility for stenting applications in peripheral vessels such as the carotid arteries, or to the surfaces of a stent graft, generally any artificial tubular-shaped prosthesis that is designed to replace a blood vessel or to create a new prosthetic functional wall inside of a damaged blood vessel. Other stent structures which may be suitably employed with the coatings of the present invention include bifurcation-type stents, designed to provide support at the branching of two vessels, for example as described in U.S. Pat. No. 6,099,560 or 6,051,020.

The invention, together with additional features and advantages thereof, may best be understood by reference to the following description taken in conjunction with the accompanying illustrative drawing.

### BRIEF DESCRIPTION OF THE DRAWINGS

FIG. 1 is a schematic view of an uncoated corrugated ring stent of the prior art;

FIG. 1a is a perspective view illustrating an individual support ring of the multiple rings arrayed in series along a longitudinal axis of an uncoated corrugated ring stent of the prior art, as illustrated in FIG. 1;

FIG. 2 is a schematic view further illustrating the pattern of the support structure of an uncoated ring stent of the prior art, wherein one wall of the structure of FIG. 1 has been cut along its longitudinal axis, and the stent has been unrolled onto a flat surface to illustrate the pattern of the support structure;

FIG. 2a is a cross-sectional view of the generally rectangular width and thickness of the stent strut taken at section A-A′ in FIG. 2;

FIG. 3 is a schematic view illustrating the stent coating process according to the principles of the invention;

FIG. 4 is a schematic view illustrating the application of the polymer solution of the present invention to the sides and top of the stent support structure;

FIG. 5 shows a cross-section view similar to that of FIG. 2A, taken along lines A-A′ of FIG. 2 after coating according to a first embodiment of the invention;

FIG. 6 shows a cross-section view similar to that of FIG. 2A, taken along lines A-A′ of FIG. 2 after coating according to a second embodiment of the invention;

FIG. 7 is a graphical representation illustrating the cumulative release of a potent derivative of Tranilast from a thicker island of coating (Curve B) and a thinner island of coating (Curve A) according to the present invention;

FIG. 8 is a graphical representation illustrating cumulative drug release from two stents containing the same total weight of a potent tranilast derivative, but having a high polymer to weight ratio (A), or low polymer to weight ratio (B).

FIG. 9 is a schematic plan view illustrating the mounting of a coated stent, produced in accordance with the teachings of the present invention, to the distal portion of an angioplasty balloon catheter for percutaneous insertion and deployment at a diseased vessel site;

FIG. 10 is a cross-sectional view of the deployed coated stent of the invention implanted in a vessel wall;

FIG. 11 illustrates a section of the stent of the present invention, with a coating selectively placed on the surface of the stent;

FIG. 12 is a view similar to that of FIG. 11, showing a section of the stent of the present invention with multiple islands of coating placed on the surface of the stent;

FIG. 13 is a view similar to those of FIGS. 11 and 12, showing a section of the stent of the present invention with multiple islands of different coating formulations placed on the stent surface; and

FIG. 14 is a view similar to those of FIGS. 11-13, showing a section of the stent of the present invention with a selectively placed coating formulation and a topcoat placed over the coating formulation.

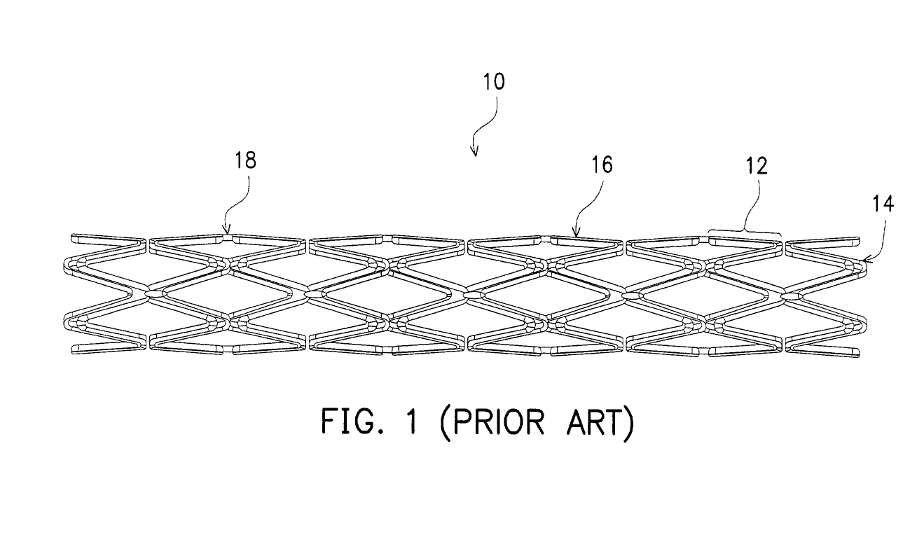

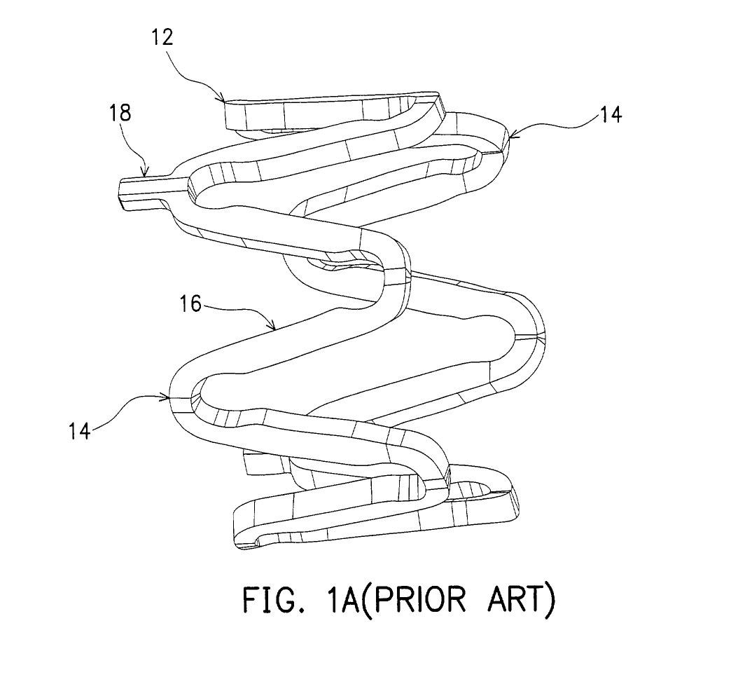

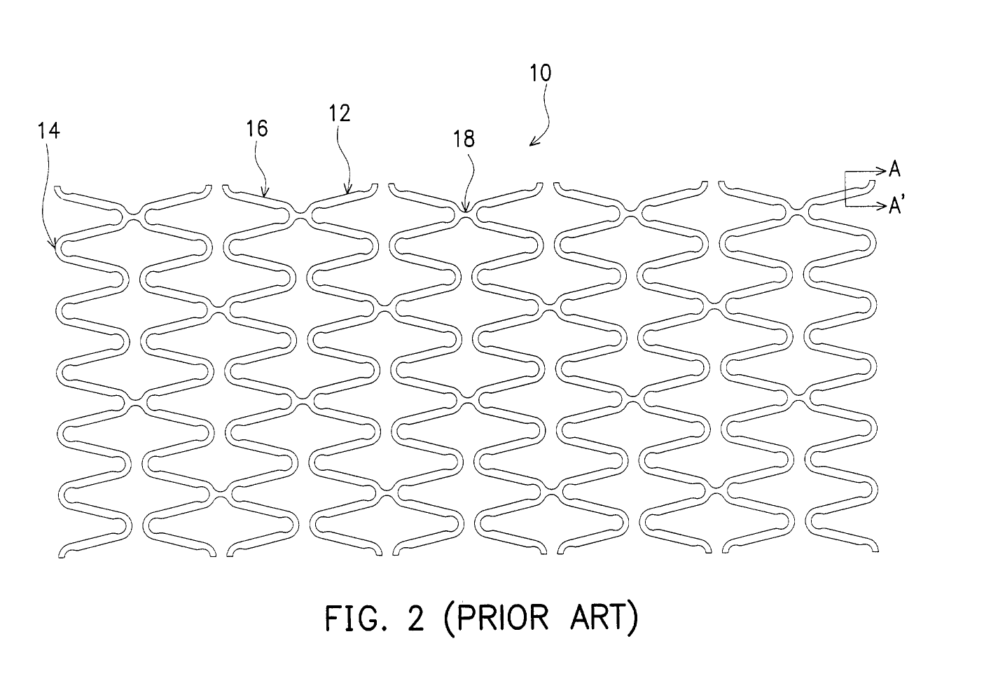

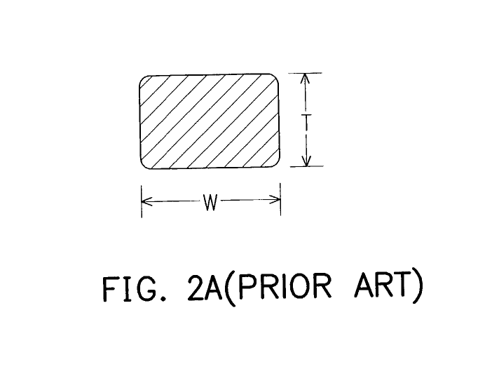

### DESCRIPTION OF EMBODIMENTS

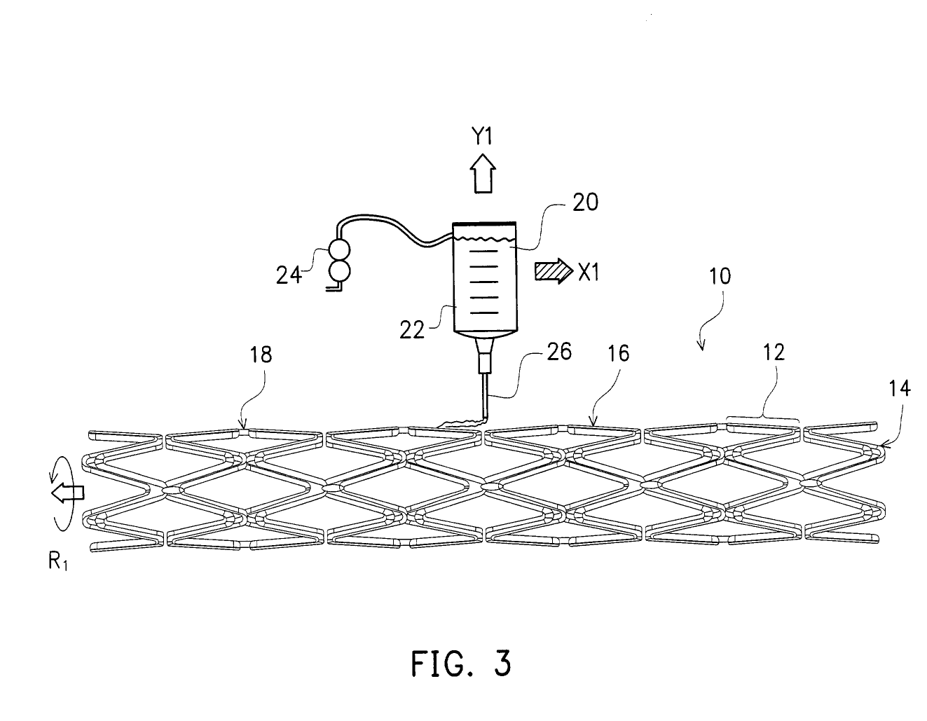

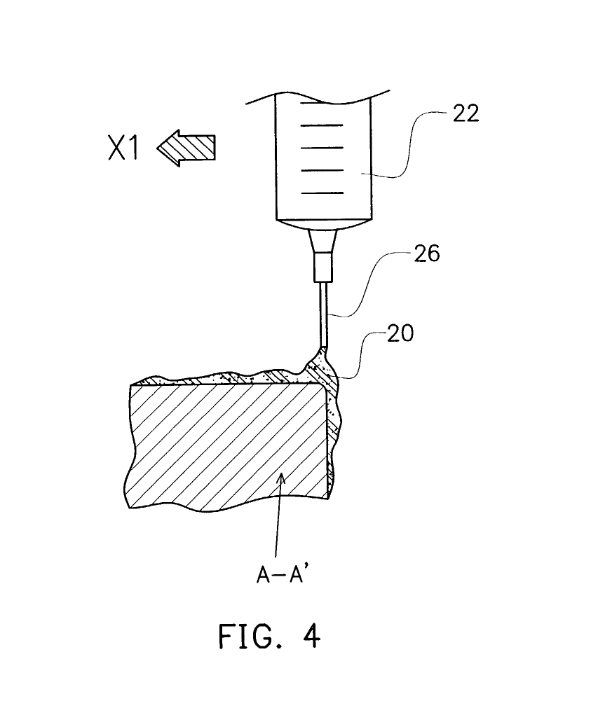

Referring now more particularly to the drawings, FIG. 3 is a schematic illustration of the stent coating process according to the invention. A polymer solution 20 is made by dissolving a polymer in a compatible solvent. At least one bioactive agent is added to the solution, either as a suspension or in solution using the same solvent or a different solvent. The completed mixture is placed in a pressurizable reservoir 22. Connected to the reservoir is a fluid pressurization pump 24. The pressurization pump 24 may comprise a compressor, a peristaltic pump, a positive displacement syringe pump, or any other source of pressure capable of urging the solvent mixture to move at a programmed rate through a solution delivery tube 26. The fluid pressurization pump 24 is under the control of a microcontroller (not shown), as is well known in the field of precision dispensing systems. For example, such a microcontroller may comprise 4-Axis Dispensing Robot Model numbers I&J500-R and I&J750-R available from I&J Fisnar Inc, of Fair Lawn, N.J., which are controllable through an RS-232C communications interface by a personal computer, or precision dispensing systems such as Automove A-400, from Asymtek, of Carlsbad, Calif. A suitable software program for controlling an RS232C interface may comprise the Fluidmove system, also available from Asymtek Inc, Carlsbad, Calif. Attached to the reservoir 22, for example, at the bottom of the reservoir 22, is the solution delivery tube 26 for delivery of the solvent mixture to the top surface of the stent. The pressurizable reservoir 22 and delivery tube 26 are mounted to a moveable support (not shown) which is capable of moving the solvent delivery tube in small steps such as 0.2 mm per step, or continuously, along the longitudinal axis of the stent as is illustrated by arrow X1. The moveable support for the pressurizable reservoir 22 and delivery tube 26 is also capable of moving the tip (distal end) of the delivery tube 26 closer to the stent surface or up away from the stent surface in small steps as shown by arrow Y1. The uncoated stent is gripped by a rotating chuck contacting the inner surface of the stent at least at one end. Axial rotation of the stent can be accomplished in small degree steps, such as 0.5 degree per step, to reposition the uppermost surface of the stent structure for coating by the delivery tube by attachment of a stepper motor to the chuck as is well known in the art. If desirable, the stent can be rotated continuously. The method of precisely positioning a low volume fluid delivery device is well known in the field of X-Y-Z solvent dispensing systems and can be incorporated into the present invention. The action of the fluid pressurizing pump, X1 and Y1 positioning of the fluid delivery tube, and R1 positioning of the stent are typically coordinated by a digital controller and computer software program, such that the precisely required amount of solution is deposited wherever desired on the surfaces of the stent, whereupon the solvent is allowed to escape, leaving a hardened coating of polymer on the stent surfaces. Typically, the viscosity of the solvent mixture is adjusted by varying the amount of solvent in the range from 2 centipoise to 2000 centipoise, and can be typically 300 to 700 centipoise. Alternatively, the delivery tube can be held at a fixed position and, in addition to the rotation movement, the stent is moved along its longitudinal direction to accomplish the coating process.

The X-Y-Z positioning table and moveable support may be purchased from I&J Fisnar. The solution delivery tube preferred dimensions are preferably between 18-21 gauge stainless steel hypotubes mounted to a suitable locking connector. Such delivery tubes may be obtained from EFD Inc of East Providence, R.I. See EFD's selection guide for Special Purpose Tips. The preferred tips are reorder #'s 5118-1/4-B through 5121-1/4-B “Burr-free passivated stainless steel tips with ¼″ length for fast point-to-point dispensing of particle-filled or thick materials”, reorder #'s 51150VAL-B “Oval stainless steel tips apply thick pastes, sealants, and epoxies in flat ribbon deposits”, and reorder #'s 5121-TLC-B through 5125-TLC-B “Resists clogging of cyanoacrylates and provides additional deposit control for low viscosity fluids. Crimped and Teflon lined”. A disposable pressurizable solution reservoir is also available from EFD, stock number 1000Y5148 through 1000Y 5152F. An alternate tip for use with the invention is a glass micro-capillary with an I.D. of about 0.0005 to 0.002 inch, such as about 0.001 inch, which is available from VWR Catalog No. 15401-560 “Microhematocrit Tubes”, 60 mm length, I.D. 0.5-0.6 mm. In the laboratory, the tubes are further drawn by pulling the tubes longitudinally under a Bunsen burner to achieve the desired I.D. for precise application of the polymer/drug/solvent mixture. The programmable microcontroller to operate the stepper motor, and XYZ table is available is available from Asymtek, Inc. It is within the scope of the invention to use more than one of the fluid dispensing tube types working in concert to form the coating, or alternately to use more than one moveable solution reservoir equipped with different tips, or containing different viscosity solutions or different chemical makeup of the multiple solutions in the same process to form the coating. The chuck and stepper motor system may be purchased from Edmund Scientific of Barrington, N.J.

Typically, as described above, the coating (polymer or non-polymer) of the present invention is applied directly onto the outside support surface(s) of the stent, which may or may not cover the entire or a portion(s) of the inside surface(s) of the stent depending on how the above described coating system of the present invention is programmed. Alternatively, the coating or coating mixture can also be applied directly onto the inside surface of the stent. A thin delivery tip penetrates through one or more of the cut out areas in the laser cut wall of the stent structure, and thereby applies the coating mixture directly onto the inside surfaces at desired areas. If the stent has a large enough dimension, a thin delivery tip can be inserted into the stent along the longitudinal axis of the stent for the purpose of applying coating to the inside surfaces.

The polymer for use in the invention includes, but not limited to, poly(D,L-lactic acid), poly(L-lactic acid), poly(D-Lactic acid), e-Caprolactone, ethylvinyl hydroxylated acetate (EVA), PVA, PEO, Ethylene Vinyl Alcohol (EVOH), and co-polymers thereof and mixtures thereof, dissolved in chloroform, or acetone, xylene, or other suitable solvents. These polymers all have a history of safe and low inflammatory use in the systemic circulation.

A non-polymer coating such as a potent derivative of tranilast which has been ionically bound to the metal stent surface can also be used in the present invention.

The bioactive agent is taken from one of a group of Diarylamide derivatives as described in an article entitled Synthesis and Structure—Activity Relationship of Diarylamide Derivatives as Selective Inhibitors of the Proliferation of Human Coronary Artery Smooth Muscle Cells, authored by Ogita et al. and published in Bioorganic & Medicinal Chemistry Letters 11 2001, pp. 549-551 (hereinafter “the Ogita reference”). The formulae of these derivatives of Tranilast are shown below:

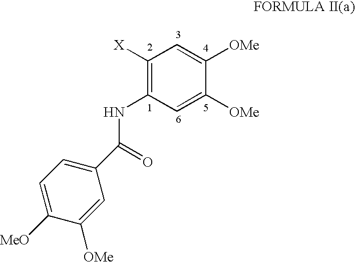

where X is —H, —CN, —CO₂H, —CO₂Et, —CONH₂, —CONHMe, —CONMe₂, or

a compound selected from the group consisting of a diarylamide derivative of the formula:

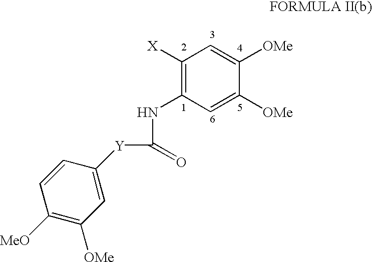

where X is —CO₂Et and Y is —CH₂— or —CH═CH—, or

a diarylamide derivative of the formula:

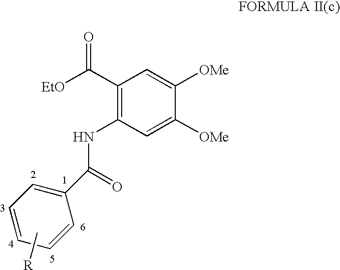

Where R is -3,4-(OMe)₂, -4-OMe, -2-OMe, -3-OMe, -4-OAc, -3,4-(OAc)₂, -3-OMe-4-OAc, -3-NO₂-4-OH, -3-NH₂-4-OH, -3,5-(OMe)₂-4-OAc, -3,4,5-(OAc)₃, -3,4,5-(OMe)₃, -2,3,4-(OMe)₃.

Or, a compound selected from the group consisting of a diarylamide derivative of the

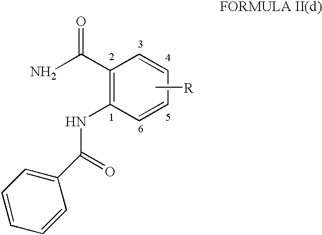

Where R is —H, -4,5-F₂, -5-NO₂, -5-NH₂, -5-Me, -4-Cl.

These drugs are potent derivatives and analogs of Tranilast, a drug which exhibits mildly antiproliferative effects in humans when taken orally. However, contrary to the teachings of U.S. Pat. No. 5,733,327 to Igaki et al., it has been discovered that tranilast does not exhibit sufficient potency to be effective to consistently reduce restenosis in the coronary overstretch injury model in pigs. The Ogita reference describes compounds with similar structure but with much higher potency than tranilast for inhibition of smooth muscle cell proliferation. The compounds actually appear to enhance endothelial cell regeneration. Thus they provide an ideal bioactive agent for production of a drug delivery stent system with marked restenosis reduction and faster healing of the vessel wall. A first example of a suitable Tranilsast derivative for use in the present invention is Formula II(a), where X=CO₂Et or CONH₂, a second suitable example is Formula II(b), where X=CO₂Et and Y=CH₂, and a third suitable example is Formula II(c), where R₂=3,4-OMe)₂, 3,4,5-OMe)₃, or 2,3,4-Ome)₃. Shown below are the chemical formulae for three suitable examples of diarylamide derivatives of the invention:

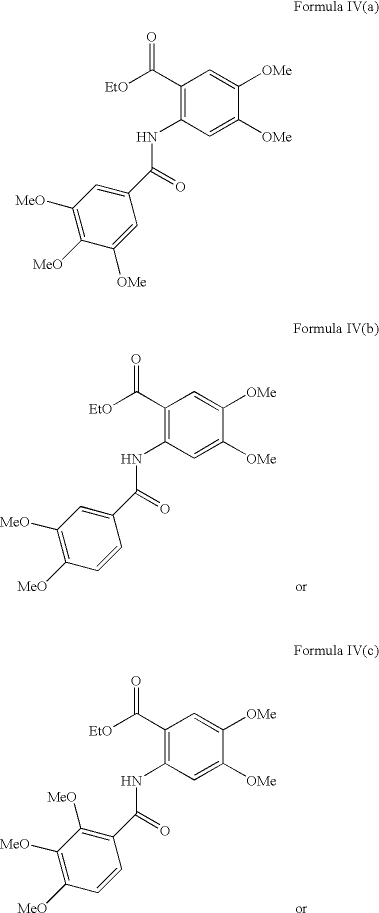

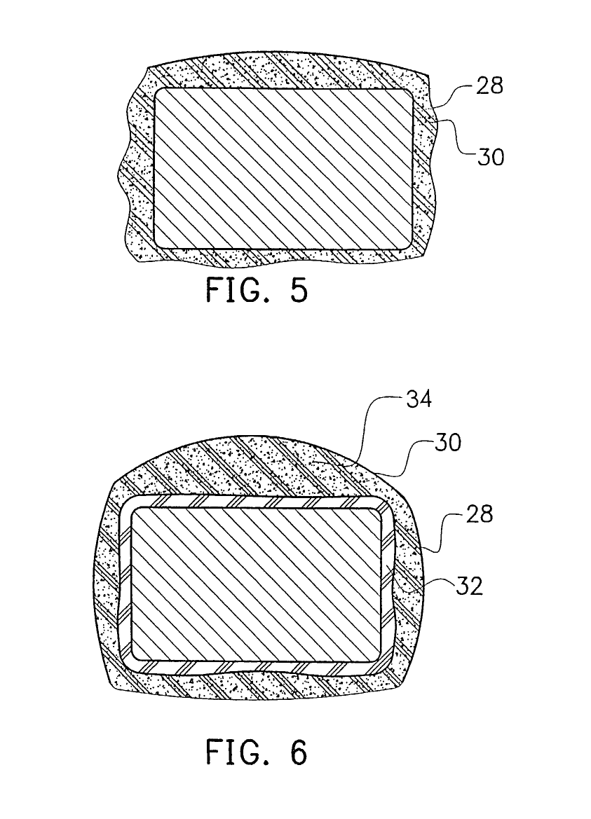

Using the coating system as described, it is feasible to coat all of the top, side, and inside surfaces of the stent bu adjusting reservoir pressure, mixture viscosity, and delivery time in each area to which a coating is applied. By the careful selection of a suitable ratio of solvent to polymer, the viscosity of the solution can be adjusted such that some of the solution will migrate down the sides of the strut and actually inhabit the bottom surface before solidifying, as shown in FIG. 5. By controlling the dwell time of the delivery tube close to the edge of the stent, the amount of polymer coating the edges or bottom of the stent can be increased or reduced.

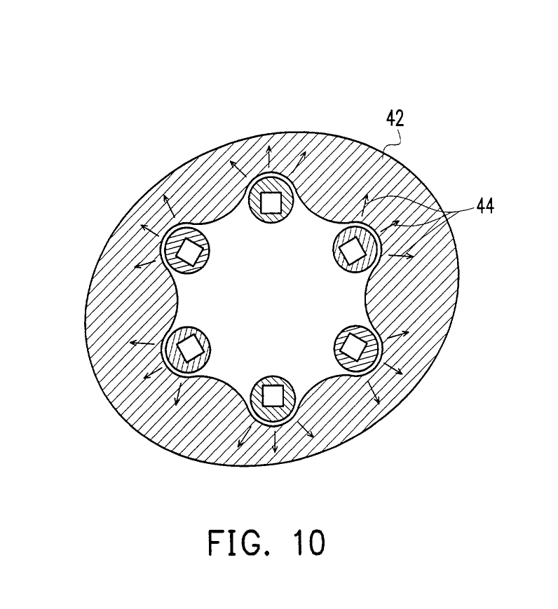

It should be appreciated however, with reference to FIG. 10, that it is not always desirable to coat the inside surfaces of the stent. For example, coating the inside surface of the stent increases the crimped delivery profile of the device, making it less maneuverable in small vessels. And, after implantation, the inside surfaces are directly washed by the flow of blood through the stent, causing any drug released on the inside surface to be lost to the systemic circulation. Therefore, an embodiment is as shown in FIG. 5, with the bulk of the cured polymer 28 and agent 30 deployed on the outside circumference (i.e. the top surface of the strut as seen in this drawing) of the stent supports, and secondarily on the sides. Only a minimum amount of polymer 28 and agent 30 is deployed on the inside surfaces of the stent (i.e. the bottom surface as seen in this drawing). If desirable, it is also possible to have at least a portion of the inside surfaces of the stent uncoated or exposed.

It is also within the scope of the present invention to produce a completely biodegradeable stent using the coating system of the current invention. This may be accomplished by making a tubular preform in the shape of the stent to be formed, using an open-top “C-shaped” cross-section channel into which the dispensing system may deposit the polymer. The preform is open at its outside diameter so that the polymer may be deposited into the preform, typically using one pass, but also possibly multiple passes of the dispensing tube, while creating uniform edges and bottom of the stent structure wherein the polymer is constrained by the preform. The preform is chosen from a material which is soluble in a solvent which will not dissolve the bio-degradable stent. After the polymer has been deposited and solvent of the polymer solution has evaporated, the assembly may be placed in the solvent which dissolves the preform to free the completed stent structure. A typical material for the preform is sucrose, which may be molded into the desired preform shape using standard injection molding techniques. A typical solvent for the preform is water. Alternately, the preform could be mechanically separated from the preform to release the stent structure.

In FIG. 6 is shown a second embodiment of the invention where water soluble heparin in crystalline form has been incorporated into the coating. The heparin crystals 34 are micronized to a particle size of approximately 1-5 microns and added in suspension to the polymer solution 20. In contrast to the procedure for making the first embodiment, in this embodiment a primer coat of pure polymer and solvent 32 is first applied to the stent surfaces using the coating system of the invention. The solvent is allowed to evaporate, then a second layer of polymer is applied containing the bioactive agent 30 and heparin 34. Suitable forms of heparin are those of crystalline form that exhibit bioactivity in mammalian hosts when applied according to the process of the invention, including heparin salts (i.e. sodium heparin and low molecular weight forms of heparin and their salts). Upon deployment of the drug delivering stent into the vessel wall, as seen in FIG. 10, the heparin crystals near the surface of the coating of cured polymer 28 begin to dissolve, increasing the porosity of the polymer. As the polymer slowly dissolves, more heparin and bioactive agent are released in a controlled manner.

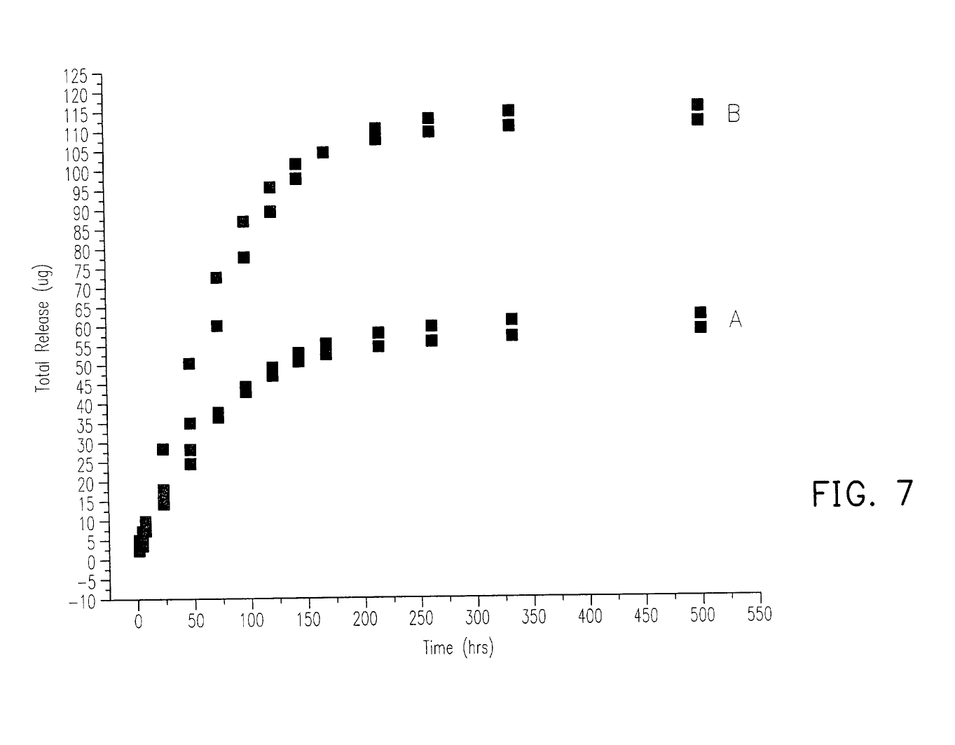

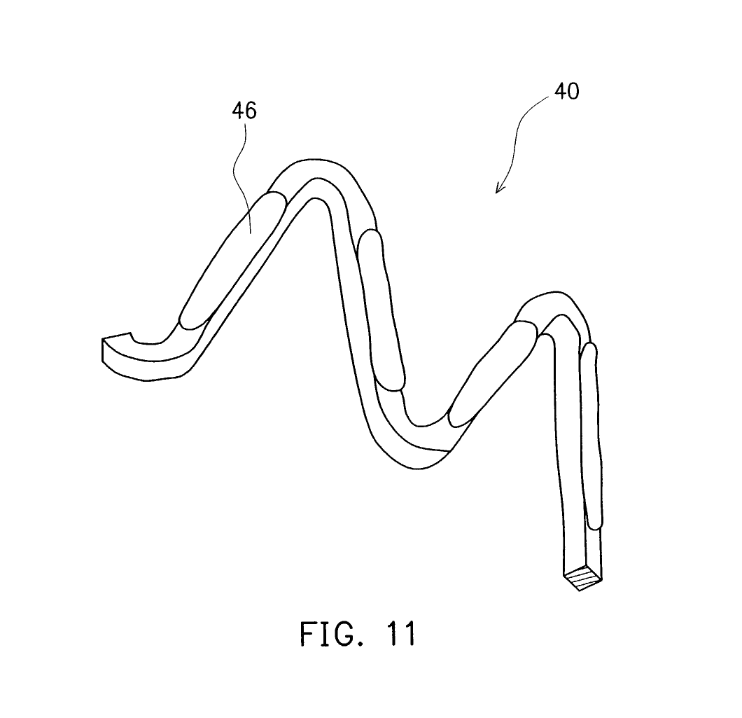

FIG. 7 shows release of the potent tranilast derivative of Fig IV(a) from two samples, each of stents made with a 10 micron thick coating (A) and 20 micron thick coating (B) where the drug polymer ratio is approximately 50% drug to 50% polymer by weight in each case. This demonstrates that drug release rates from the islands of coating as shown in FIGS. 11-13 can be tailored to have higher or lower cumulative drug release by varying the thickness of the coatings.

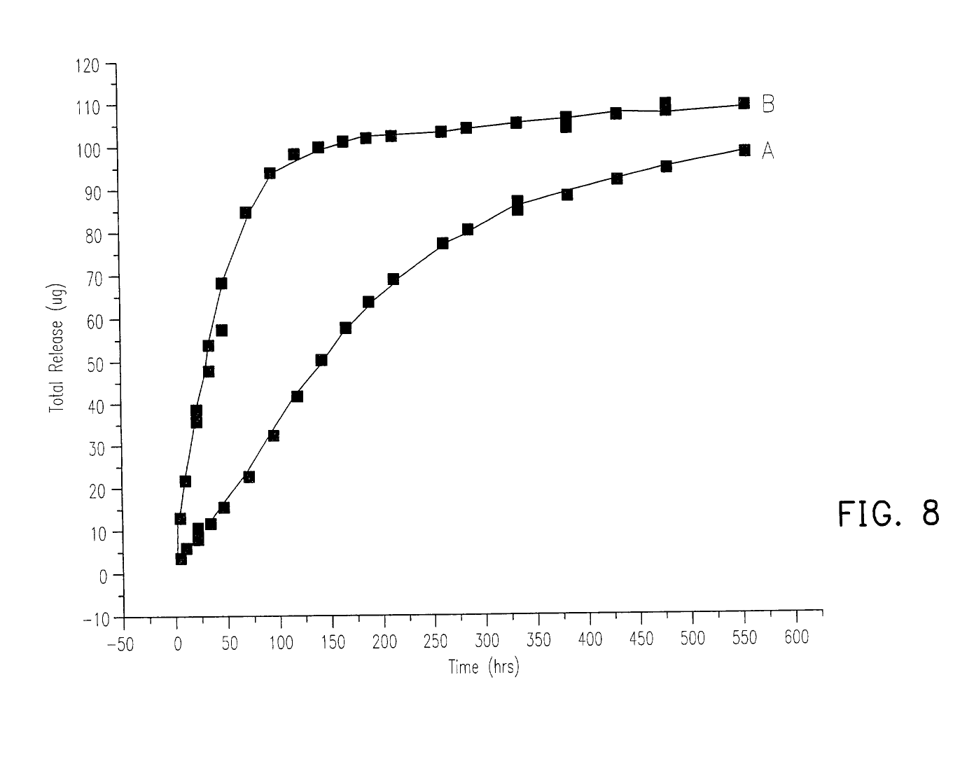

FIG. 8 shows release of the potent tranilast derivative of Fig IV(b) of the present invention with 105 micrograms of the derivative being released from a stent coating of 40% drug to 60% polymer by weight (A), and from a stent coating (B) of 60% drug to 40% polymer weight ratio. These curves demonstrate the ability to deliver the drug at faster or slower rates by varying the ratio of drug to polymer in the coating.

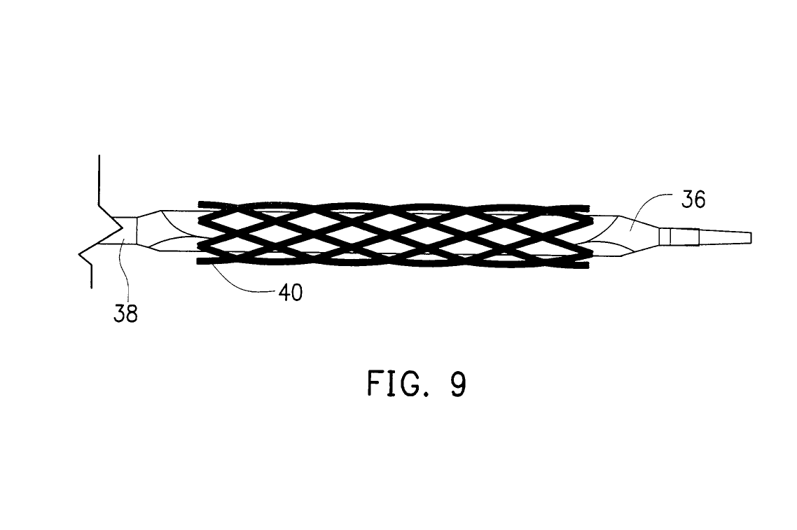

In FIG. 9, there is shown the distal end of a coronary angioplasty catheter including a high pressure balloon 36 and a distal shaft 38, wherein a coated stent 40 has been crimped. The coatings produced according to the present invention have high mechanical integrity to withstand the stresses of mechanical crimping to the balloon and subsequent re-expansion and deployment into the vessel wall. The coatings are flexible and adherent enough to remain firmly attached to the stent support structure during the crimping and delivery process.

FIG. 10 shows the first embodiment of the invention deployed in the vessel wall. As shown, most of the polymer-containing drug can be oriented in the direction of the vessel wall 42, minimizing drug loss to the flowing blood, and providing more efficient delivery of the drug directly into the injured tissue, as illustrated by the arrows 44 which trace the diffusion pathways for the drug. Thus, little medication is wasted, and the drug delivery stent 40 has a low overall profile compared to other designs when mounted on the delivery balloon 36.

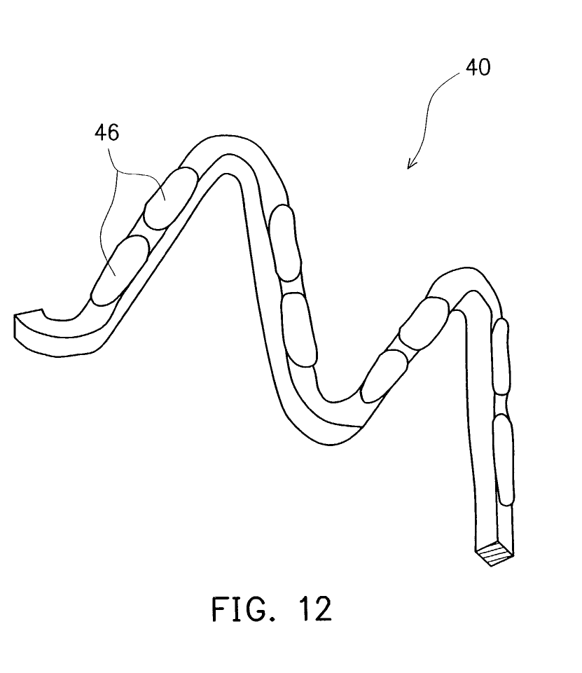

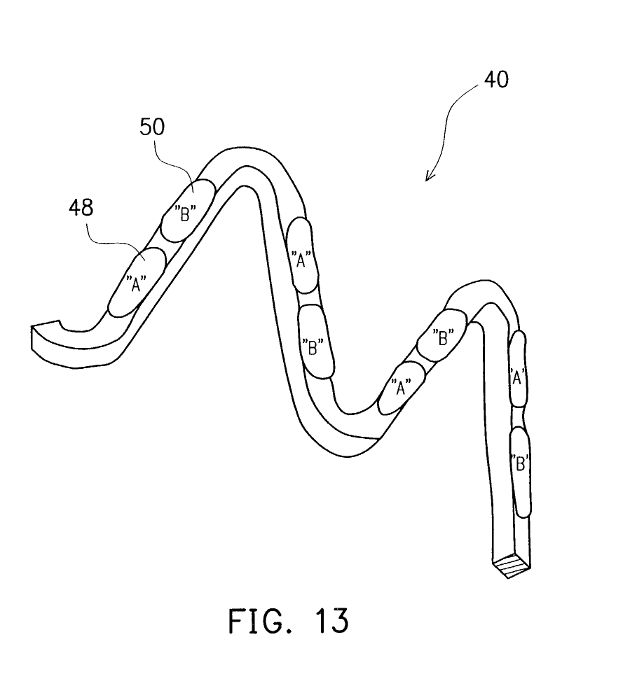

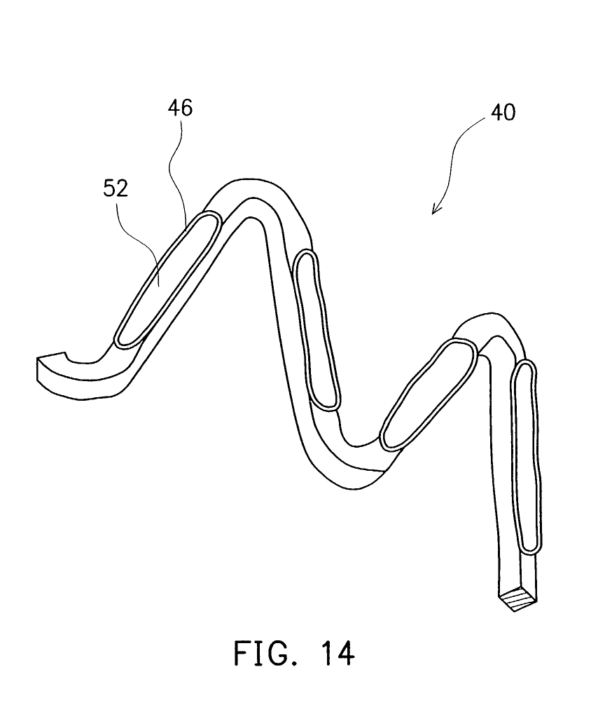

In FIG. 11, a coated section 46 is placed onto the stent surface in a selective manner. The depth of the coated section may correspond to the volume of bioactive coating to be available for presentation to the tissue, as described in conjunction with FIG. 7 above. It may be advantageous to restrict the coating from certain areas, such as those which incur high strain levels during stent deployment. A uniform primer coat may be first placed on the stent surface to promote adhesion of the coating which contains the bioactive agent, such as a primer coat 32 shown in FIG. 6. The primer coat may be applied by using any of the methods as already known in the art, or by the precision dispensing system of the invention. It is also within the scope of the invention to apply a primer coat using a different polymer material, such as parylene (poly(dichloro-para-xylylene)), ethylene vinyl alcohol, or any other material which exhibits good adhesion to both the base metal substrate and the coating which contains the bioactive agent. Parylene (poly(dichloro-para-xylylene)) may be deposited via sputter coating or vapor deposition techniques as is well known in the art (See U.S. Pat. No. 6,299,604). As shown in FIG. 12, multiple islands or sections 46 of the same coating, each having the same thickness, or different thickness, are within the scope of the invention, and may release the same or different total amounts of drug, at the same, or at different delivery rates, as discussed in conjunction with the FIGS. 7 and 8. Further, as shown in FIG. 13, different coating formulations 48, 50, with different agents or combinations of bioactive agents may be likewise applied on the same stent 40 using the coating process as described. Optionally, a topcoat 52 may be applied over the coatings, for example to control dissolution of the bioerodable polymer underneath, or to control agent diffusion rate from a biodurable polymer 46 as shown in the embodiment of FIG. 14. In one embodiment of the present invention, islands of a coating containing heparin are formed on inside surface(s) of a stent and an anti-proliferation coating containing the drugs of the present invention as described above are formed on outside surface(s) of the stent.

The following are some examples of how to fabricate devices within the scope of the current invention:

### EXAMPLE 1

100 mg poly(DL-lactide) (Sigma) was dissolved into 2 mL acetone at room temperature. 3 mg tranilast derivative of Fig. IV(a) was placed into a 3 mL septum-top v-vial, 300 μL lactide solution was added, and the drug was allowed to dissolve at room temperature. A glass syringe was used to withdraw and apply 10 μL of the drug containing lactide solution to the outer strut surfaces of a 10 band (15 mm length) stent cut from 2 mm diameter, 316LVM stainless steel hypotube. Evaporation of the solvent resulted in a uniform, drug containing single polymer layer on the stent. This method was also used to precision dispense 15 μL of the same polymer solution to the stent. This resulted in a single layer coating on the stent strut top and sides. In vitro drug release was conducted by placing the coated stents into 2 mL pH 7.4 phosphate buffered saline solution preserved with 0.05% (w/v) sodium azide and maintained at 37° C. Sampling was periodically conducted by withdrawing the total buffer volume for drug measurement while replacing solution with a similar volume of fresh buffer (infinite sink). Tranilast derivative was quantified by UV measurement and comparison to known standards.

### EXAMPLE 2

100 mg poly(DL-lactide) is dissolved into 2 mL chloroform at room temperature. 3 mg tranilast derivative of Fig. IV(b) was placed into a vial and 300 μL lactide solution added. A microprocessor-controlled syringe pump was used to precision dispense 10 μL of the drug containing lactide solution to the stent strut top surface to form a single layer of drug containing polymer.

### EXAMPLE 3

An adhesion optimizing primer coat was prepared by dissolving 100 mg poly(DL-lactide) with 2 mL acetone. 15 μL of this solution was dispensed onto the stent strut top and side surfaces and the solvent was allowed to dry before dispensing a single layer of drug containing polylactide solution to the stent strut.

### EXAMPLE 4

2 mg of the tranilast derivative of Fig. IV(c) was placed into a vial and 200 μL poly(DL-lactide)/acetone (50 mg/mL) was added. One-half of this solution was removed and added to 0.9 mg finely powdered sucrose. A syringe was used to dispense 15 μL of the sucrose-containing solution to stent strut outer surfaces. As a control, 15 μL of the solution without sucrose was dispensed in a similar manner to outer strut surfaces of identical stents. In vitro drug elution was determined using infinite sink techniques and quantitative UV analysis.

Accordingly, although exemplary embodiments of the invention has been shown and described, it is to be understood that all the terms used herein are descriptive rather than limiting, and that many changes, modifications, and substitutions may be made by one having ordinary skill in the art without departing from the spirit and scope of the invention. It is intended that the scope of the invention be limited not by this detailed description, but rather only by the claims appended hereto.

What is claimed is:

1. A stent comprising:
an expandable stent body having a generally tubular shape;
a plurality of support surfaces on said stent body upon which a coating is applied, said coating containing one or more bioactive agents disposed therein, wherein at least one of said bioactive agents is a potent derivative of Tranilast.

2. The stent of claim 1, wherein said derivative is a diarylamide derivative of Tranilast.

3. The stent of claim 2, wherein said diarylamide derivative is taken from the group: 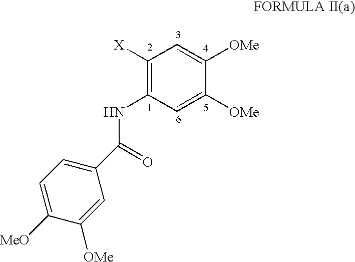
where X is —H, —CN, —CO₂H, —CO₂Et, —CONH₂, —CONHMe, —CONMe₂, or
a compound selected from the group consisting of a diarylamide derivative of the formula: 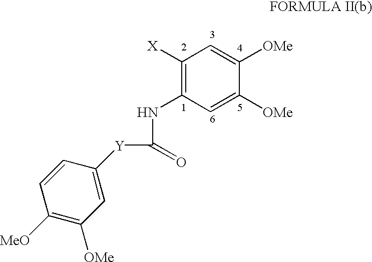
where X is —CO₂Et and Y is —CH₂— or —CH═CH—, or
a diarylamide derivative of the formula: 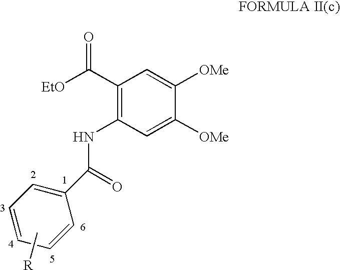
Where R is -3,4-(OMe)₂, -4-OMe, -2-OMe, -3-OMe, -4-OAc, -3,4-(OAc)₂, -3-OMe-4-OAc, -3-NO₂-4-OH, -3-NH₂-4-OH, -3,5-(OMe)₂-4-OAc, -3,4,5-(OAc)₃, -3,4,5-(OMe)₃, -2,3,4-(OMe)₃.
Or, a compound selected from the group consisting of a diarylamide derivative of the formula:

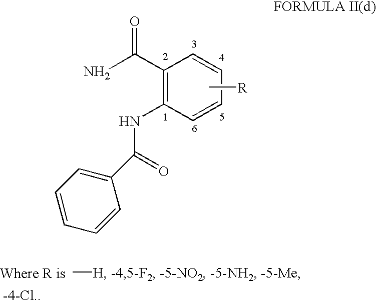

4. The diarylamide derivative of claim 3, Formula II(a), where X=CO₂Et or CONH₂

5. The diarylamide derivative of claim 3, Formula II(b), where X=CO₂Et and Y=CH₂

6. The diarylamide derivative of claim 3 Formula II(c), where R₂=3,4-(OMe)₂, 3,4,5-(OMe)₃, or 2,3,4-(OMe)₃

7. The stent as recited in claim 1, wherein said coating is a polymer coating which contains said bioactive agent or agents.

8. The stent of claim 7, wherein said polymer coating is a bioerodeable coating.

9. The stent as recited in claim 7, wherein said polymer is selected from the group including Poly(D,L-lactic acid), Poly(L-lactic acid), Poly(D-lactic acid), Poly(glycolic acid), e-Caprolactone, or copolymers thereof, or mixtures thereof.

10. The stent as recited in claim 7, wherein the thickness of the coating on one of said plurality of support surfaces is different than the thickness of the coating on another one of said plurality of support surfaces, to thereby cause a proportionate increase or decrease in bioactive agent delivery in various regions of the stent.

11. The stent as recited in claim 7, wherein the polymer coating is applied only on selected portions of said support surfaces, forming islands of the polymer coating on the stent.

12. The stent as recited in claim 10, wherein the bioactive agent contained in one of said islands is different from the bioactive agent contained in another one of said islands.

13. The stent as recited in claim 11, wherein the thickness of one of said islands is different from the thickness of another one of said islands.

14. The stent as recited in claim 7, wherein at least one of said bioactive agents is in a crystalline form.

15. The stent as recited in claim 7, wherein the coating contains heparin in crystalline form as a bioactive agent.

16. The stent of claim 7, wherein said polymer coating acts as a drug storage reservoir for bioactive agents disposed therein, and wherein at least one of said bioactive agents is an antiplatelet, fibrinolytic, or thrombolytic agent in soluble crystalline form.

17. The stent of claim 16, wherein said antiplatelet, fibrinolytic, or thrombolytic agent is continuously eluted from the drug storage reservoir.

18. The stent as recited in claim 16, wherein said antiplatelet, fibrinolytic, or thrombolytic agent is heparin, aspirin, hirudin, ticlopadine(sp), eptifibatide, urokinase, streptokinase, tissue plasminogen activator (TPA), or abciximab, or a mixture thereof.

19. The stent of claim 1, wherein the stent contains a derivative of Tranilast of sufficient potency to be effective in controlling restenosis when incorporated in a polymer coating with a thickness of not more than 25 microns.

20. The stent as recited in claim 1, further comprising a primer polymer coating between said support surfaces and said coating.

21. The stent as recited in claim 20, wherein the primer polymer coating is formed substantially of poly(dichloro-para-xylylene).

22. The stent as recited in claim 1 further comprising a topcoat on said coating.

23. The stent as recited in claim 7, wherein said polymer is non-erodable polymer selected from the group including poly(vinyl alchohol) (PVA), EVA, EVOH, or copolymers thereof, or mixtures thereof.

24. A vascular stent containing a bioactive agent in an amount effective for reduction of restenosis, wherein the bioactive agent comprises one or more of the following related compounds: 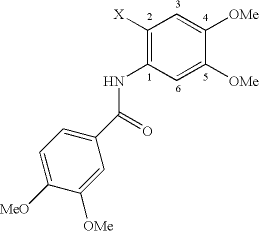
where X is —H, —CN, —CO₂H, —CO₂Et, —CONH₂, —CONHMe, —CONMe₂, or, 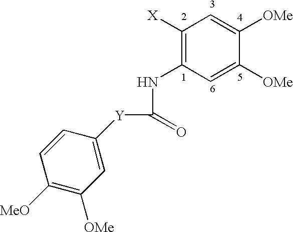
where X is —CO₂Et and Y is —CH₂— or —CH═CH—, or 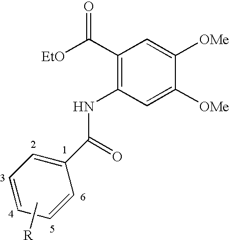
Where R is -3,4-(OMe)₂, -4-OMe, -2-OMe, -3-OMe, -4-OAc, -3,4-(OAc)₂, -3-OMe-4-OAc, -3-NO₂-4-OH, -3-NH₂-4-OH, -3,5-(OMe)₂-4-OAc, -3,4,5-(OAc)₃, -3,4,5-(OMe)₃, -2,3,4-(OMe)₃., or 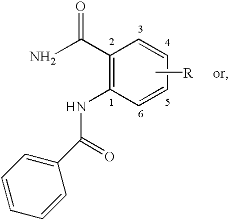
Where R is —H, -4,5-F₂, -5-NO₂, -5-NH₂, -5-Me, -4-Cl.

25. The vascular stent of claim 24, wherein the bioactive agent is

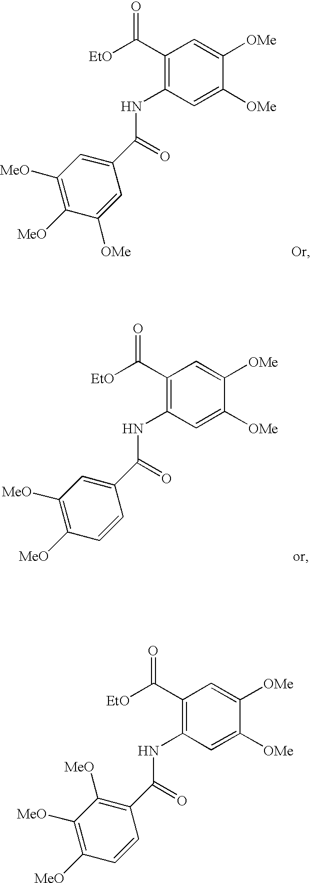

26. The stent as recited in claim 7, wherein the support surfaces comprises outside support surfaces and inside support surfaces, the coating comprises an outside coating applied on the outside support surfaces and an inside coating applied on the inside support surfaces, wherein the inside coating forms one or more islands containing heparin in crystalline form.

27. A vascular stent containing a bioactive agent effective for the reduction of restenosis where the bioactive agent is a potent analogue or derivative of tranilast, which selectively controls smooth muscle cell proliferation, while not interfering to any substantial degree with proliferation of endothelial cells.

28. The stent as recited in claim 27, wherein the bioactive agent is a diarylamide derivative of tranilast.

29. A vascular stent having a stent support structure, and containing two biologically active agents in a coating on a surface of the stent support structure, wherein first one of said two agents is present in an effective quantity for control of restenosis, and second one of said two agents comprises an antiplatelet, fibrinolytic, or thrombolytic agent, wherein said antiplatelet, fibrinolytic, or thrombolytic agent, and is released for the same duration as the anti-restenosis agent.

30. The vascular stent as recited in claim 29, wherein the second agent is heparin, aspirin, hirudin, ticlopadine(sp), eptifibatide, urokinase, streptokinase, tissue plasminogen activator (TPA), or abciximab.

31. A fully bioerodable stent which is fabricated by deposition of a mixture of polymer and solvent on a preform, said polymer coating containing one or more bioactive agents.

32. The bioerodable stent of claim 31, wherein the preform is made of sucrose.

33. The bioerodable stent of claim 31, wherein the preform is soluble in a solvent which does not dissolve the polymer coating

34. The bioerodable stent of claim 31, wherein, after applying the polymer coating on the preform, the stent is placed in said solvent which dissolves the preform to free a completed stent structure.

35. The bioerodable stent of claim 31, wherein at least one of said bioactive agents is potent derivative of Tranilast.

36. The bioerodable stent of claim 31, wherein one of said bioactive agents is an antiplatelet, fibrinolytic, or thrombolytic agent in soluble crystalline form.

37. The bioerodable stent of claim 31, wherein one of said bioactive agents is an antiplatelet, fibrinolytic, or thrombolytic agent in soluble crystalline form.

38. The bioerodable stent of claim 31, wherein at least one of said bioactive agents is heparin, aspirin, hirudin, ticlopadine(sp), eptifibatide, urokinase, streptokinase, tissue plasminogen activator (TPA), or abciximab, or a mixture thereof.

39. The bioerodable stent of claim 31, wherein said bioactive agents are continuously eluted.

40. The bioerodeable stent of claim 35, wherein said potent derivative of Tranilast is a diarylamide derivative of Tranilast.

41. A method of coating a stent comprising an expandable stent body having a generally tubular shape, said stent body comprising a plurality of outside support surfaces and corresponding inner surfaces, said method comprising:
applying a polymer coating to said outside support surfaces, said coating including one or more bioactive agents and being applied from a delivery tube coupled to a pressurized source; and
coordinating said tube's position along a longitudinal axis of said stent, and rotation of the stent about said longitudinal axis using a programmable controller.

42. The method as recited in claim 41, wherein the coating is applied from a distal end of the delivery tube, and a vertical height of the distal end of said tube is coordinated by said controller.

43. The method as recited in claim 41, wherein said step of applying a polymer coating is accomplished in a single pass.

44. The method as recited in claim 41, wherein the position of the tube is moved in small increments to sequentially apply the polymer solution across all of the interconnected outside surfaces of the stent support structure.

45. The method as recited in claim 41, wherein the position of the tube is moved continuously to sequentially apply the polymer solution across all of the interconnected outside surfaces of the stent support structure.

46. The method as recited in claim 41, wherein the thickness of the polymer coating is controlled by adjusting one or more of the following: the speed of rotation of the tube, the dwell time of the distal end of the tube at specific areas on the stent, the pressure of the pressurized source, the speed of movement of the distal end of the delivery tube, the vertical height of the distal end, the viscosity of the polymer solution, or the types of polymer and solvent.

47. The method as recited in claim 41, wherein the step of applying a polymer coating to said outside support surfaces is controlled, so that the thickness of the polymer coating on the inner surfaces is significantly smaller than thickness of the polymer coating on the outside support surfaces.

48. The method as recited in claim 41, wherein the step of applying a polymer coating to said outside support surfaces is controlled, so that the polymer coating forms isolated islands on the outside support surfaces.

49. The method as recited in claim 48, wherein one of said islands contains a bioactive agent different from that contained in another one of said islands.
